(19) United States
(12) Patent Application Publication
(10) Pub. No.: US 2017/0319380 A1
(43) Pub. Date: Nov. 9, 2017

(54) WEARABLE APPARATUS FOR COOLING

(71) Applicant: Pocket Sky OG, Wein (AT)

(72) Inventors: Michael Geyer, Wien (AT); Mark Wallerberger, Wien (AT)

(21) Appl. No.: 15/519,950

(22) PCT Filed: Oct. 20, 2015
(86) PCT No.: PCT/AT2015/050262

§ 371 Date: Apr. 18, 2017

(30) Foreign Application Priority Data
Oct. 20, 2014 (AT) A 50750/2014

(51) Int. Cl. A61F 7/02; A41D 20/00; A61F 7/00

(52) U.S. Cl. CPC A61F 7/02; A61F 7/0053; A41D 20/005; A61F 2007/0035; A61F 2007/0096; A61F 2007/0228; A61F 2007/0064

(57) ABSTRACT

A portable cooling device with a wristband (17) and a cooler arranged on the wristband (17), in which the cooler is designed as an air flow generator (1) for an airflow directed in an exhaust direction of the cooler (1), wherein the airflow generator (1) is arranged in a holder (11) which is fastened or formed to the wristband (17) and which at least has a extension (10) protruding in the exhaust direction, said extension (10) at least located sectionally within the airflow or enclosing the airflow at least sectionally. The invention thus provides a portable cooling device which can be designed to be extremely small and compact and can thus be worn inconspicuously. The device according to the invention is also robust and has a lower power consumption compared to conventional devices.

The invention relates to a portable cooling device with a wristband and a cooler arranged on the wristband according to the preamble of claim 1.

At warm ambient temperatures, cooling of the body is desired. If cooling of the ambient air itself is not given, cooling of the skin surface can be performed. For this purpose, devices such as fans or the like are known which achieve a cooling effect by means of an airflow on the skin surface. Furthermore, a portable cooling device with a wristband and a cooler arranged on the wristband is known, wherein the cooler is implemented as a Peltier element. Peltier elements, however, are characterized by a high energy requirement, so that portable devices with Peltier elements have only short operating times and comparatively long charging times. In addition, the Peltier element must be used in close contact with the skin surface, which is sometimes felt to be unpleasant.

It is therefore the object of the invention to provide a portable cooling device with a wristband and a cooler arranged on the wristband, which has a smaller power consumption than known devices, and thus longer operating times at the same charging times. In addition, the cooling device according to the invention should be inconspicuous to wear, and can also be used with appropriate clothing, for example when working on a computer with a wrist resting on the tabletop.

These objects are achieved by the features of claim 1. According to the invention, in a portable cooling device with a wristband and a cooler arranged on the wristband, the cooler is designed as an airflow generator for an airflow directed in an exhaust direction of the cooler, the airflow generator being attached in a mount on the wristband, which at least has an extension showing in the exhaust direction, and said extension is at least located within or at the airflow section-wise or surrounds the airflow at least section-wise.

According to the invention, an airflow generator arranged in a holder is provided, wherein the exhaust direction of the airflow is oriented inward with the wristband closed, ie. with the wristband applied in the direction of the skin surface. Supporting a suitable air flow in the exhaust direction is essential for the function of the cooling device, which is ensured according to the invention by means of an extension showing in the exhaust direction, and said extension located at least partially within or at the airflow or enclosing the airflow at least partially. In this way, with the wristband applied—as will be explained in more detail—a cooling or exhausting region for the airflow is provided, whereby a maximum cooling effect is achieved by blowing the cooling airflow against the skin surface. The wristband is applied in the wrist area in the usual manner, the airflow generator being located above the pulse point of the wrist, that is the area in which the pulse can be felt. Cooling of pulse points has a great influence on the subjective heat sensation of the human being. Due to the blowing of air, the wearer of the inventive wristband feels subjectively cooler without the entire body having to be cooled. One of the most sensitive pulse points is thereby at the hand root, which is utilized by the cooling device according to the invention.

However, the holder of the airflow generator can also be provided with a cover on its outwardly oriented side when the wristband is closed, said cover providing air-permeable regions through which air can be sucked into the cooling device. These air-permeable areas can be achieved either by perforations in an air-impermeable cover plate, or by air permeability of the material of the cover used. The holder of the airflow generator can also be provided with a cover on its outwardly oriented side when the wristband is closed, with holder and said cover forming at least one lateral suction opening as an air-permeable region. The sucked-in air thus enters the cooling device through the at least one lateral suction opening from a direction oriented essentially perpendicular to the clear cross-section of the closed wristband and is subsequently deflected into the exhaust direction. The cover provided for this purpose supports this deflection of the sucked-in air and also ensures it when the cooling device according to the invention is worn under a long-sleeved shirt or a blouse for example. The cover also protects the airflow generator from diminishing the air flow generation by the clothing worn over the cooling device or when the forearm is lying on a tabletop or the like.

The air-flow generator can be designed, for example, as a piezo-fan. These coolers are based on the piezoelectric effect and are also known as “Dual Piezoelectric Cooling Jets” (DCJ). They have two thin metal plates with piezoelectric elements, which move at frequencies of several hundred Hertz to kilohertz when an alternating voltage is provided and so produce a high-speed airflow which is delivered via a nozzle. Similar to a bellows, ambient air is sucked in and released as cooling air. Piezo fans are preferably used which are operated at frequencies above 20 kHz so that they are not perceptible to the human ear.

When such piezo fans are used, it is proposed that the at least one extension is nozzle-shaped. The at least one nozzle-shaped extension is directed towards the surface of the skin, so that the skin surface is streaked or circulated with cooling air. In this case, the nozzle effect can be utilized, in which strongly moved air entrains the surrounding air and thus multiplies the fan effect.

However, the air-flow generator can also be designed as an axial fan, which is mounted in a breakthrough of a mount, which is designed as a bearing plate, with an axis of rotation that is perpendicular to the bearing plate. The cover is preferably designed as a cover plate spanning the axial fan. This cover plate favors the exclusive suction of air through the lateral suction openings and increases the throughflow speed of the sucked-in air at the same rotational speed of the axial fan. The lateral suction openings can also be covered by slats which protect the axial fan from the penetration of harmful small parts and, if appropriate, also repel spraying water, but allow the inflowing air to pass unimpeded.

The extensions can also be designed as webs running perpendicular to the clear cross-section of the closed wristband. Such an embodiment promotes the adjustment of an exhaust direction of the air to be exhausted perpendicular to the clear cross-section of the closed wristband. In this case, the webs can also be designed curved, in particular in the form of webs which are closer to one another in their middle regions than in their outer end regions. This embodiment favors the emission of the heated air. It has also been found to be particularly advantageous if the extensions are designed to be thermally conductive, for example of metallic material, since they are thus able to absorb heat from the surface of the skin which is in contact with the skin and to release it to the airflow.

The airflow generator can be put into operation by means of a corresponding control element or by merely closing the wristband. However, in order to avoid a habituation effect of the carrier and thus a reduced cooling effect, it is proposed that the wristband is provided with a control unit, which is connected to the air-flow generator for the realization of switching-on and switching-off cycles. Each time the air-flow generator is switched on again, the cooling effect is again subjectively perceived intensively. An air-flow generator, in contrast to a Peltier element, has the advantage that the cooling effect can be activated or deactivated without delay immediately after switching on or off, so that fast control is possible. In addition, the current requirement for the air flow generator can be reduced by means of switch-on and switch-off cycles and the operating time can be increased significantly. Furthermore, the wristband can also be provided with pulse and/or temperature sensors, which are connected to the control unit. These sensors measure the skin and/or the ambient temperature and can use the control units to adjust cooling times, cooling time and cooling phases in such a way that an optimal subjective cooling effect is achieved. Moisture meters, position sensors and the like can also be provided and their data can be used to optimize the cooling effect. These sensors can be installed in the holder, in the extensions of the holder or in other parts of the wristband.

Of course, the wristband provided according to the invention can also serve as a carrier for other devices, for example for watches with additional functions (“smart watches”), solar cells or portable communication devices. The power supply provided for the airflow generator can also serve as a power supply for the additional device, or the power supply provided for the additional device as a power supply for the air flow generator.

### BRIEF DESCRIPTION OF THE DRAWINGS

The invention is explained in more detail below with reference to exemplary embodiments by means of the appended figures.

FIG. 1 shows a perspective view of an embodiment of the cooling device according to the invention with axial fan,

FIG. 2 shows a perspective view of an embodiment of the cooling device according to the invention during use,

FIG. 3 is a sectional view through the embodiment of the cooler according to FIG. 1,

FIG. 4 shows a perspective view of a further embodiment of the cooling device according to the invention,

FIG. 5 shows a sectional view of an embodiment of the cooling device according to the invention in the operating position for explaining the generated air flow,

FIGS. 6a and 6b are perspective views of the cooling device according to the invention with different embodiments of the spacers,

FIG. 7 shows a sectional view through an embodiment of the cooler,

FIGS. 8a and 8b are illustrations for explaining possible embodiments of damping elements,

FIG. 9 is a perspective view of a further embodiment of the cooling device according to the invention with a smart watch,

FIG. 10 shows a perspective view of a further embodiment of the cooling device according to the invention with additional sensors,

FIG. 11 is a representation of the cooler for explaining a possible embodiment of the suction openings,

FIG. 12 shows a sectional view through a further embodiment of a cooling device according to the invention with a piezo-fan,

FIG. 13 shows a sectional view of an embodiment according to the invention with a piezo-fan and a possible embodiment of a nozzle-shaped extension,

FIG. 14 shows a sectional view of a further embodiment of a nozzle-shaped extension when using a piezo-fan,

FIG. 15 shows a sectional view of an embodiment according to the invention with a piezo-fan and a further embodiment of the nozzle-shaped extensions,

FIG. 16 is a sectional view in the direction A-A of FIG. 15,

FIG. 17 shows a sectional view of an embodiment according to the invention with a piezo-fan and a further embodiment of a nozzle-shaped extension with a downwardly oriented suction opening,

FIG. 18 shows a sectional view of an embodiment according to the invention with a piezo-fan and a further embodiment of nozzle-shaped extensions and downwardly oriented suction openings.

Reference is first made to FIG. 1, which shows a perspective view of an embodiment of the cooling device according to the invention with a wristband 17 and a holder 11 fastened or formed to the wristband 17, which is designed as a bearing plate in the exemplary embodiment shown. The holder 11 forms a part of the peripheral region of the wristband 17 when the wristband 17 is closed. In the holder 11 designed as a bearing plate, an air flow generator 1 embodied as an axial fan which is mounted rotatably, the axis of rotation of the axial fan and thus the exhaust direction of the generated air flow being oriented perpendicularly to the bearing plate.

The holder 11 is provided with a cover 4 on its outwardly oriented side with the wristband 17 closed, which forms at least one lateral suction opening 2 with the holder 11. For this purpose, the holder 11 and the cover 4 are made of a sufficiently rigid material. In the exemplary embodiment shown, the cover 4 is designed as a cover plate spanning the axial fan.

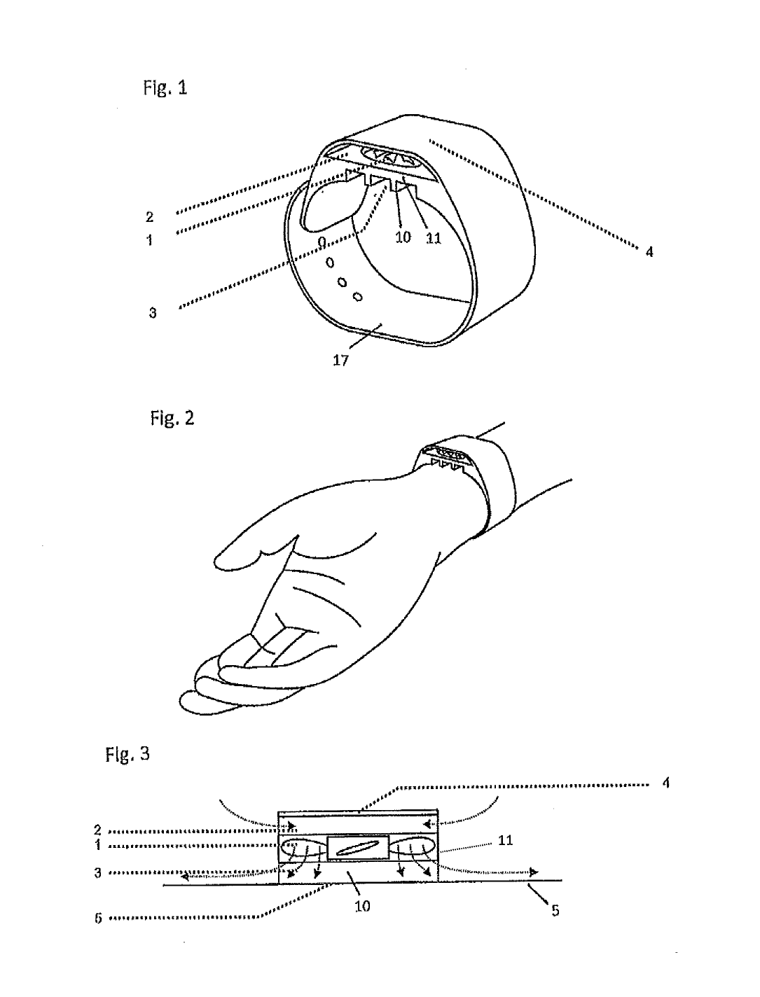

On the side of the holder 11, which is oriented inwards when the wristband 17 is closed, protruding extensions 10 in the exhaust direction are arranged. In the exemplary embodiment shown, the extensions 10 are designed as two webs running perpendicular to the clear cross-section of the closed wristband 17, which are integrally formed on the holder 11. When the wristband 17 is applied, the extensions 10 are also used as spacers to ensure sufficient spacing of the holder 11 from the skin surface and to form a cooling space for the generated airflow below the holder 11, as can be seen from FIG. 2.

FIG. 2 shows a perspective view of an embodiment of the cooling device according to the invention during use, wherein it can be seen that the wristband 17 is applied in the wrist area in a conventional manner, the airflow generator 1 being located above the pulse point of the wrist, i.e. the area where the pulse can be felt. As already mentioned, the cooling of pulse points has a great influence on the subjective heat sensation of the human being. The carrier of the cooling device according to the invention feels subjectively cooler by the inflated air, without the entire body having to be cooled. One of the most sensitive pulse points is thereby located at the hand root, which is utilized by the cooling device according to the invention. The extensions 10 lie close to the wrist during the use of the cooling device according to the invention and ensure the spacing of the holder 11 with the airflow generator 1 from the skin surface 5 (see also FIG. 3). Preferably, the extensions 10 can be designed to be thermally conductive so that they can dissipate heat from the skin surface 5 and can deliver it to the bypassing airflow.

With reference to FIG. 3, which shows a sectional view through the embodiment of the cooler according to FIG. 1, the air flow which is produced by the cooler is explained. The axial fan conveys the air flow from the suction side to the pressure side parallel to the axis of rotation of the axial fan. However, due to the cover 4 spaced apart from the holder 11 which forms at least one lateral suction opening 2 between the cover 4 and the holder 11, the sucked air enters the cooling device through the at least one lateral suction opening 2 perpendicular to the clear cross section of the closed wristband 17 and is subsequently deflected into the rotational axis direction by the axial fan and ejected into the exhaust direction, which is parallel therewith, to the pressure side. On the side of the holder 11, which is oriented inward when the wristband 17 is closed, the airflow thus produced is ensured by the protrusions 10 protruding from the holder 11 in the exhaust direction according to the invention. In this way, when the wristband 17 is applied, a cooling area for the airflow between the holder 11 and the skin surface 5 is provided, whereby a maximum cooling effect is achieved by blowing vertically to the skin surface 5 with the cooling airflow and, on the other hand, lateral exhausing openings 3 are provided by spacing the holder 11 from the skin surface 5, through which the heated air can leave the cooling device according to the invention.

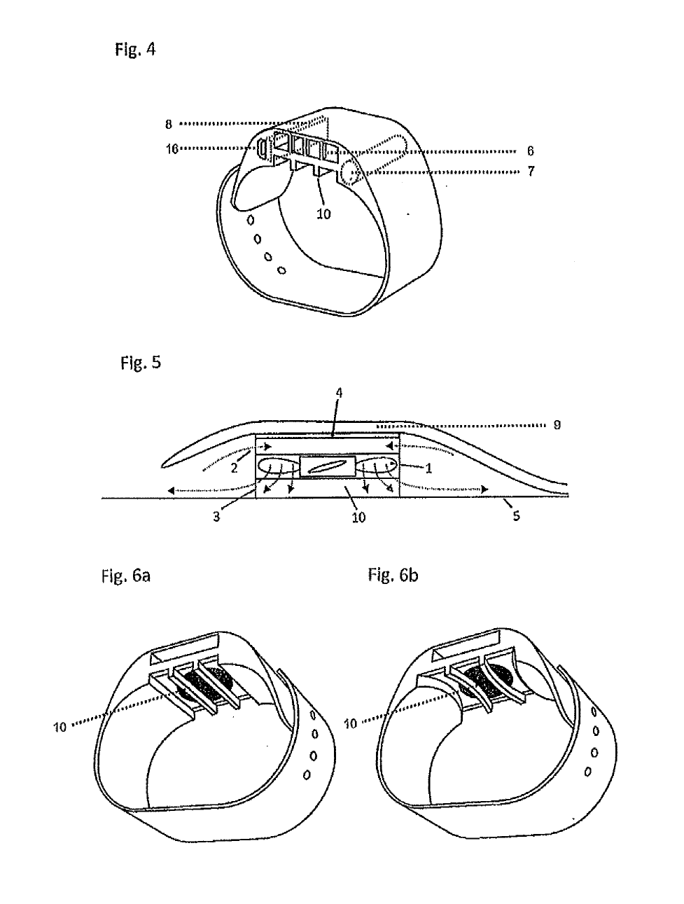

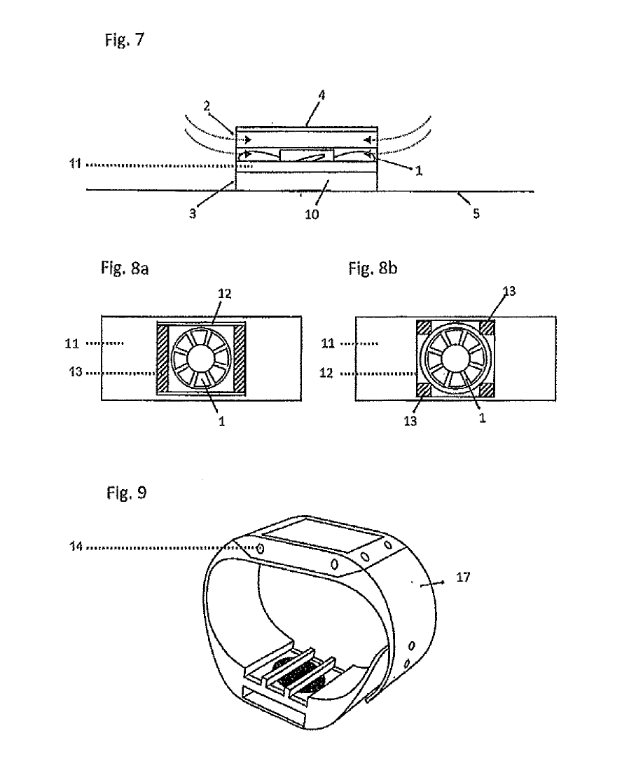

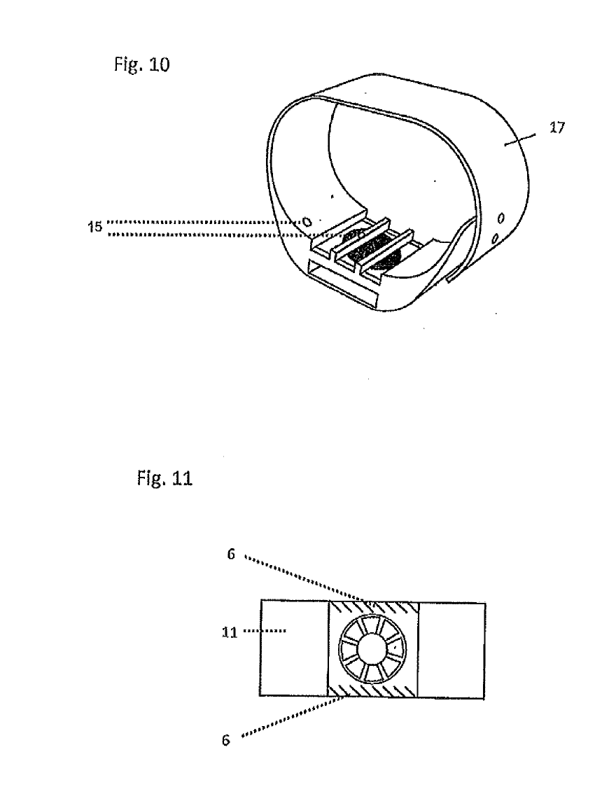

FIG. 4 shows a perspective view of a further embodiment of the cooling device according to the invention, the lateral suction opening 2 being provided with lamellas 6 spanning the suction opening 2. Furthermore, a control unit 7 arranged in the wristband 17 as well as a power supply 8 with a charging socket 16 can be seen. The power supply 8 is designed, for example, as a rechargeable battery via a micro-USB. The control unit 7, the airflow generator 1 and the power supply 8 are connected to one another via electrical conductors (not shown). The control unit 7 can be designed approximately such that it controls the switching-on and switching-off cycles of the air-flow generator 1 and thus avoids a habituation effect of the carrier and the associated reduction in the subjective cooling effect. In addition, the current requirement for the air flow generator 1 is reduced and the operating time can be extended considerably. The control unit 7 can also perform further functions, as will be explained in more detail below.

The generated air flow within an embodiment of the cooling device according to the invention with an axial fan in the operating position is explained with reference to the FIG. 5, the cooling device being supported below a shirt sleeve 9. It can be seen how the cover 4 protects the axial fan from an influence which hinders the rotational movement of the axial fan through the clothing worn over the cooling device. The deflection of the sucked air from a suction direction perpendicular to the clear cross-section of the closed wristlet 17 into a cooling air flow oriented perpendicular to the skin surface 5 is effected by the cooperation of the cover 4, the axial fan and the extensions 10.

FIGS. 6a and 6b show perspective views of the cooling device according to the invention with different embodiments of the extensions 10. As already mentioned, the extensions 10 can be designed as webs running perpendicular to the clear cross-section of the closed wristband 17, two such webs being shown, for example, in FIG. 6a. Such an embodiment helps to adjust the exhaust direction of the air to be exhausted perpendicular to the clear cross-section of the closed wristband 17. In this case, the webs can also be curved, as can be seen in FIG. 6b. The curvature is realized in such a way that the webs lie closer to each other in their middle regions than in their outer end regions so that the clear cross-section of the exhaust openings 3 widens from the middle regions to the end regions of the webs. The expanded cross-section of the exhaust openings 3 promotes the exhaustion of the exausted air because the flow velocity of the air in the direction of the exhaust openings 3 decreases and the expanding volume reduces backflow effects. The extensions 10 designed as webs are located at least section-wise within the airflow.

FIG. 7 shows a sectional view through an embodiment of an air flow generator embodied as an axial fan. It can be seen that the axial ventilator, at least along its peripheral area, which faces at least one suction opening 2, protrudes beyond the holding surface 11 designed as a bearing plate on its suction side. This arrangement increases the suction volume for the sucked-in air and thus the quantity of cooling air at the same rotational speed of the axial fan.

Regarding the axial fan, designs are available which are distinguished by high running stability and quiet operation. However, in order to absorb any possible vibrations of the axial fan and to avoid the transmission of vibrations to the wrist of the wearer, elastic damping elements 13 can be provided, which are arranged in the contact regions between a housing 12 accommodating the axial fan and the mount 11. FIGS. 8a and 8b show possible embodiments of these damping elements 13, which can be formed, for example, by thin rubber lamellas and can be designed, for example, as edge strips on the edge (FIG. 8a) or as corner lamellas (FIG. 8b). These damping elements 13 also absorb impact forces which are produced when being worn by the hand. In this way, the bearing of the axial fan is protected from damage.

As already mentioned, the wristband 17 provided according to the invention can also serve as a carrier for other devices 14, for example for watches with additional functions (“smart watches”) or for portable communication devices. FIG. 9 shows, for example, the use of a cooling device according to the invention with a smart watch. The cooling device according to the invention and the additional device 14 are located on opposite sides of the wristband 17. It is thereby possible to use the same components for the cooling device and the additional device 14. For example, the power supply 8 provided for the airflow generator 1 can also serve as a power supply for the additional device 14, or the power supply provided for the additional device 14 as a power supply 8 for the airflow generator 1.

As can be seen from FIG. 10, the cooling device according to the invention can also be provided with pulse and/or temperature sensors 15, which are connected to the control unit 7. These pulse and/or temperature sensors 15 measure the skin and/or the ambient temperatures and can use the control units 7 to set cooling times, cooling duration and cooling phases in such a way that an optimum subjective cooling effect is achieved. Moisture meters, position sensors and the like can also be provided and their data can be used to optimize the subjective cooling effect. These sensors, as well as the pulse and/or temperature sensors 15 mentioned, can be installed in the extensions 10, in the holder 11 or in other parts of the wristband 17.

FIG. 11 shows a further embodiment of the lamellas 6 spanning the lateral suction opening 2. The lamellas arranged parallel to one another in the form of a group protect the airflow generator 1, which is designed as an axial fan, against the penetration of harmful small parts and, in the case of a suitable embodiment, also against splashing water, but allow the inflowing air to pass unimpeded.

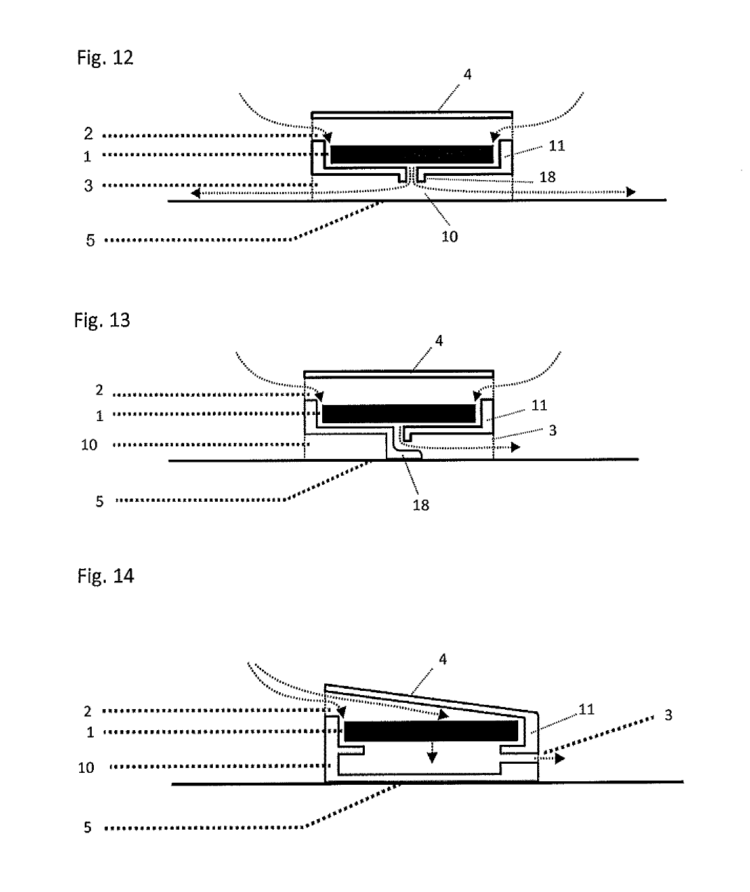

A further embodiment of a cooling device according to the invention with an air-flow generator 1 designed as a piezo-fan is explained with reference to FIGS. 12 to 18. The piezo fan is arranged in a holder 11 which, on its outwardly oriented side, is provided with a cover 4 which forms at least one lateral suction opening 2 with the holder 11. The holder 11 can also be provided with a nozzle-shaped exhaust opening 18, through which the airflow generated by the airflow generator 1 emerges. On the side of the holder 11 which is oriented inwardly with the wristband 17 closed, there are arranged inwardly projecting extensions 10, which, when the wristband 17 is applied, again serve as a spacer for the skin surface 5. As a rule, in such an embodiment, the exhaust opening 18 is arranged between two of the extensions 10 so that the extensions 10 enclose the airflow at least in sections.

As shown in FIG. 13, the nozzle-shaped exhaust opening 18 can also be extended so that it itself also functions as a spacer, whereby the exhausted airflow is deflected by the jet-shaped exhaust opening 18 in such a way that the generated air flow emerges from the exhaust opening 3 parallel to the skin surface 5 and flows along the skin surface 5 in a cooling manner.

FIG. 14 shows a sectional view of a further embodiment of an extension 10 with a nozzle-shaped exhaust opening 3 which is designed in such a way that the generated air flow again emerges parallel to the skin surface 5 from the exhaust opening 3 and flows along the skin surface 5 in a cooling manner. In this embodiment, the extension 10 completely encloses the generated airflow.

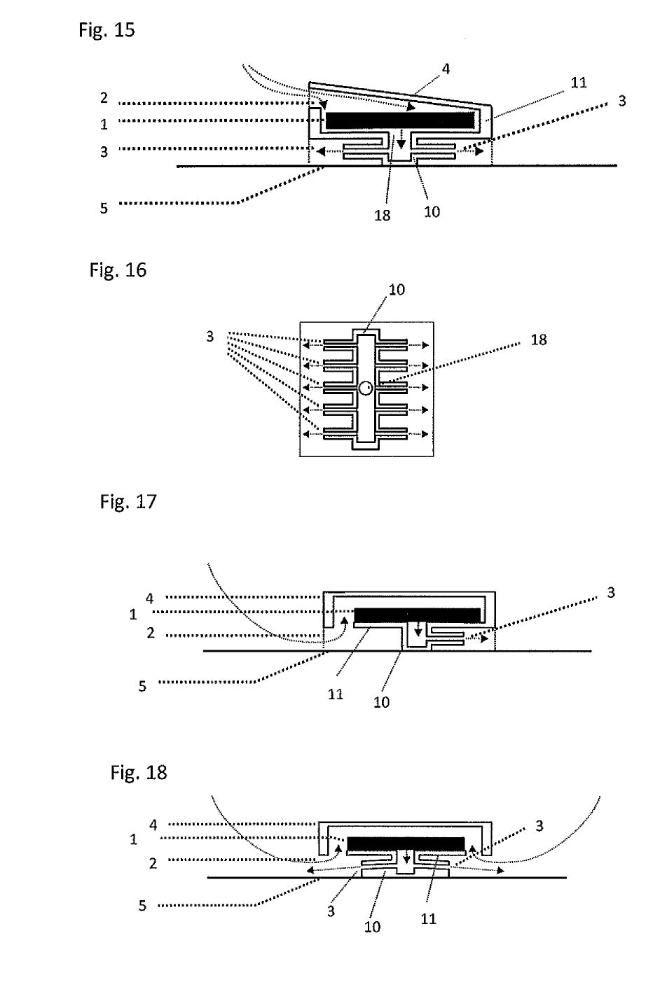

FIGS. 15 and 16 show cross-sectional views of an embodiment according to the invention with an air-flow generator 1 designed as a piezo-fan and a further embodiment of the extensions 10 which have a plurality of nozzles 3 in the exemplary embodiment shown, the generated airflow emerging from the exhaust openings 3 and flowing along the skin surface 5 in a cooling manner. Also in this embodiment, the extension 10 completely encloses the generated airflow and forms an exhaust space until the airflow emerges at the exhaust openings 3.

FIG. 17 shows a sectional view of an embodiment according to the invention with an air flow generator 1 embodied as a piezoelectric fan and a further embodiment of a extension 10 with a nozzle-shaped exhaust opening 3, where the generated airflow emerges from the exhaust opening 3 and flows along the skin surface 5 in a cooling manner. Furthermore, a further embodiment of the suction opening 2 can be seen, which is now located on the side of the mount 11, which is oriented inwards when the wristband 17 is closed. When the wristband 17 is closed, air is sucked along the skin surface 5, which enters the cooling device through the suction opening 2. As a result, it is accelerated by the air-flow generator 1, which is designed as a piezo-fan, and emerges from the cooling device again through the nozzle-shaped extension 10. The nozzle-shaped extension 10 also serves as a spacer in this case in order to ensure sufficient spacing of the holder 11 from the skin surface 5 and thus to keep the suction opening 2 free.

FIG. 18 shows an embodiment which is comparable to FIG. 17, but is symmetrical. Again, when the wristband 17 is closed, air is sucked in along the skin surface 5, which subsequently enters the cooling device through the suction opening 2. As a result, it is accelerated by the air-flow generator 1, which is designed as a piezo-fan, and emerges from the cooling device again through the nozzle-shaped extension 10. However, the nozzle-shaped extension 10 is now designed symmetrically and delivers the cooling airflow on both sides of the cooling device in opposite directions. The extension 10 also serves as a spacer in this case in order to ensure sufficient spacing of the holder 11 from the skin surface 5 and thus to keep the suction opening 2 free.

The invention thus provides a portable cooling device which can be designed to be extremely small and compact and thus can be worn inconspicuously, for example, under appropriate clothing. The device according to the invention is furthermore robust and resistant and thus is suitable for everyday use, and has a lower power consumption compared to conventional devices and thus has longer operating times.

### REFERENCE LIST

1 Airflow generator
2 Suction opening
3 Exhaust opening
4 Cover
5 Skin surface
6 Lamellas
7 Control unit
8 Power supply
9 Shirt sleeves
10 Extensions
11 Mounting bracket
12 Housing
13 Damping elements
14 Additional device
15 Pulse and/or temperature sensors
16 Charging socket
17 Wristband
18 Exhaust nozzles

1-10. (canceled)

11. A portable cooling device comprising:
a wristband; and
at least one cooler arranged on said wristband, said cooler including at least one airflow generator having configuration capable of generating an airflow directed in an exhaust direction of said cooler, said airflow generator being located in a holder associated with said wristband;
wherein said holder having at least one extension extending in the exhaust direction, and said extension being located at least partially within or adjacent the airflow or enclosing the airflow at least partially.

12. The portable cooling device according to claim 11, wherein said holder is provided with a cover which has at least one air-permeable region, said cover being on an outwardly oriented side of said holder when said wristband is in a closed orientation.

13. The portable cooling device according to claim 12, wherein said holder defines at least one suction opening adjacent said cover for communicating air to an inlet of said airflow generator.

14. The portable cooling device according to claim 13, wherein said suction opening is orientated lateral to the exhaust direction.

15. The portable cooling device according to claim 14, wherein said wristband includes lamellas spanning said suction opening.

16. The portable cooling device according to claim 11 further comprises elastic damping elements arranged in contact regions between a housing accommodating said airflow generator and said holder.

17. The portable cooling device according to claim 11, wherein said airflow generator is a piezoelectric fan.

18. The portable cooling device according to claim 11, wherein said airflow generator is an axial fan mountable in a breakthrough defined in said holder, said breakthrough is configured as a bearing plate with an axis of rotation perpendicular to said bearing plate.

19. The portable cooling device according to claim 11, wherein said extension is nozzle-shaped.

20. The portable cooling device as claimed in claim 11, wherein said extension is a plurality of extensions provided as webs running perpendicular to a cross-section of said wristband in a closed orientation.

21. The portable cooling device according to claim 20, wherein said webs are curved.

22. The portable cooling device according to claim 11, wherein said extension is configured to be heat-conducting.

23. The portable cooling device according to claim 11, wherein said wristband is provided with a control unit connected to said airflow generator, said control unit having a configuration capable of switching said airflow generator between on and off cycles.

24. The portable cooling device according to claim 23, wherein said wristband is provided with at least one sensor in communication with said control unit, said sensor being selected from the group consisting of a pulse sensor, and a temperature sensor.

25. The portable cooling device according to claim 11, wherein said extension has a configuration capable of contacting a skin surface of a wearer of said wristband.

26. The portable cooling device according to claim 11, wherein said holder includes at least one exhaust nozzle configured to direct the airflow emerging from said airflow generator.

27. The portable cooling device according to claim 26, wherein said exhaust nozzle is arranged between two of said extension so as to enclose at least a portion of the airflow.

28. The portable cooling device according to claim 26, wherein said exhaust nozzle has a configuration capable of directing the airflow emerging from an exhaust opening associated with said airflow generator parallel to a skin surface of a wearer of said wristband.
